(19) United States
(12) Patent Application Publication
(10) Pub. No.: US 2011/0280918 A1
(43) Pub. Date: Nov. 17, 2011

(54) COMPOSITION COMPRISING PHOSPHOLIPIDS IN COMBINATION WITH ASCORBIC ACID AND COSMETIC PRODUCTS COMPRISING IT

(75) Inventor: Meir Shinitzky, Herzelia (IL)

(73) Assignee: L. RAPHAEL SA, Geneve (CH)

(21) Appl. No.: 12/989,988

(22) PCT Filed: Oct. 23, 2008
(86) PCT No.: PCT/EP2008/009016

§ 371 Date: Feb. 28, 2011

(30) Foreign Application Priority Data
Apr. 28, 2008 (IL) 191123

(51) Int. Cl. A61K 8/55; A61K 8/97; A61P 3/02; A61K 8/60; A23L 1/305; A61Q 1/02; A61P 39/06; A61Q 13/00; A23J 7/00; A61K 8/64; A61Q 19/00

(52) U.S. Cl. 424/401; 514/78; 424/94.1; 514/21.9; 424/725; 514/27; 426/662; 426/648

(57) ABSTRACT

A paste composition comprising at least 25% phospholipids by weight in combination with at least 0.5% ascorbic acid by weight and a cosmetic product comprising this paste and a cosmetically acceptable carrier.

### FIELD OF THE INVENTION

The present invention relates to a paste composition comprising a high content of phospholipids in combination with ascorbic acid, to a method for its preparation, and to cosmetic and nutraceutical products based on such paste composition.

### BACKGROUND OF THE INVENTION

Phospholipids are abundant in animals and plants. The term “lecithin” is often used to denote a substance containing phospholipids, which include a complex mixture of lecithin and other phospholipids.

Due to its many beneficial properties, lecithin is used as a component in cosmetic products and also as food additive. It is used as an emulsifier, thickener, stabilizer, mild preservative, moisturizer, and emollient. Being a water-attracting agent, lecithin is used in products to help hydrate the skin and to improve the texture and ease of spread onto the skin. Cosmetically, it may be added to moisturizers, make-up, shampoos, conditioners, body washes, lip balms, and many other products. For food use, lecithin is used as a dietary supplement or as an additive for chocolate, baked goods, salad dressing, and many other prepared foods.

Lecithin is regarded as a well tolerated and non-toxic compound and has been approved by the Food and Drugs Administration (FDA), USA, for GRAS-status (Generally Recognised As Safe), making it suitable for long-term use.

Commercial lecithin is usually extracted from either egg yolk or soybeans. Purified lecithin or fractionated lecithin is obtainable by subjecting soybean lecithin or egg yolk lecithin to purification operation(s), such as solvent fractionation, extraction and/or fractionation to remove neutral oily matter as sufficiently as possible. A usual method for preparation of from egg yolk is total lipid extraction with hexane. The fraction remaining after evaporation of triglycerides under vacuum is termed “lecithin”, but it is very impure. For enrichment of the phospholipids/lecithin content, the hexane is evaporated, followed by addition of water to the total lipid extract and distillation, producing a fraction containing about 10% phospholipids. The lecithin fraction may be further purified by mixing it with acetone, in which lecithin is insoluble, thus resulting in a product that contains 20-25% phospholipids.

Egg yolk lecithin is considered superior to that originating from soybeans, but it is also considerably more expensive. The commercial price of lecithin products increases sharply with the content of phosphatidylcholine: the price of the commercially available products containing 30-40% phosphatidylcholine is about 10 times higher than that of products containing 20% phosphatidylcholine. Cosmetic products currently on the market contain no more than 2% lecithin, predominantly due to price restrictions.

Phospholipids/lecithin are used as a component of cosmetic products. U.S. Pat. No. 5,997,888 describes a cosmetic preparation in the form of a nanodispersion comprising lecithin. U.S. Pat. No. 6,080,424 discloses a cosmetic powder comprising a phospholipid phase consisting essentially of natural lecithin. U.S. Pat. No. 6,312,703 describes a pharmaceutical or cosmetic composition comprising lecithin, without the use of emulsifiers or other ingredients which are typically used as carriers for phospholipids, wherein said lecithin is a liquid crystal solid structurally deformable and shapeable into a dosage form suitable for oral or topical administration. U.S. Pat. No. 6,416,771 discloses lecithin as a component of moisturizing agents for cosmetic products. U.S. Pat. No. 6,669,932 discloses phospholipids such as egg yolk and soybean lecithin as a component of skin-whitening cosmetic material.

Ascorbic acid or Vitamin C (L-ascorbic acid) is known for its antioxidant activity and can be effectively absorbed by the skin. Thanks to its antioxidant properties, ascorbic acid is considered to confer both antioxidant and photoprotection to skin against free radical attack and UV ray damage. Over the last years, L-ascorbic acid has been incorporated into cosmetic products to provide antioxidant protection against damaging UVA and UVB rays, to help prevent premature signs of aging, such as fine lines and wrinkles, and to contribute to collagen synthesis.

Because of its importance for the cosmetic industry, it would be desirable to provide a product with increased phospholipids content, which allows incorporation of high concentrations of phospholipids in cosmetic products.

### SUMMARY OF THE INVENTION

Surprisingly, it has been found that a purified product with a high phospholipid content further containing ascorbic acid can be obtained by treating commercial egg yolk lecithin with ethanol and further treatment with ascorbic acid. After separation, a phospholipid-enriched fraction is obtained in the form of a paste also comprising ascorbic acid.

Thus, in one aspect, the present invention relates to a paste composition comprising at least about 25% phospholipids by weight in combination with at least about 0.5% ascorbic acid by weight.

Also provided is a method for the preparation of the paste composition of the invention, comprising the steps:
(i) forming an emulsion of a lipid extract in ethanol;
(ii) adding water containing ascorbic acid to said emulsion of (i), under mixing, and transferring to a separatory device, whereby three different layers are formed, the viscous bottom layer mainly consisting of phospholipids; and
(iii) collecting said bottom viscous liquid layer of (ii) and adding water thereto, thereby obtaining the desired paste composition comprising at least about 25% phospholipids and least about 0.5% ascorbic acid.

The phospholipids may be obtained from any suitable source, including, but not limited to, egg yolk and soybean. In a preferred embodiment, the starting lipid extract is from egg yolk.

The present invention further relates to cosmetic products and to nutraceutical products comprising the paste composition of the invention.

### DETAILED DESCRIPTION OF THE INVENTION

In a first aspect, the present invention relates to a is paste composition comprising at least 25% phospholipids by weight in combination with at least 0.5% ascorbic acid by weight.

In one embodiment, the paste composition comprises about 25% to about 60%, preferably about 30 to about 40%, phospholipids, by weight, and about 0.5% to about 1.5%, preferably about 1%, ascorbic acid by weight, based on the total weight of said paste composition. In one preferred embodiment, at least 50% of said phospholipids is phosphatidylcholine. The content of the compositions and products disclosed herein expressed as percent is always based on the total weight of said composition or product. The phospholipids are preferably obtained from an egg yolk lipid extract.

In a further aspect, the present invention relates to a method for the preparation of the paste composition of the invention, comprising the steps:
(i) forming an emulsion of a lipid extract in ethanol;
(ii) adding water containing ascorbic acid to said emulsion of (i), under mixing, and transferring to a separatory device, whereby three different distinct layers are formed, the viscous bottom layer consisting mainly of phospholipids; and
(iii) collecting said bottom viscous liquid layer of (ii) and adding water thereto, thereby obtaining the desired paste composition comprising at least about 25% phospholipids and at least about 0.5% ascorbic acid.

In step (i), the emulsion is formed by mixing a lipid extract with 70%-100%, preferably 95%, ethanol at a temperature in the range of 10° C.-70° C., preferably 20° C. to 60° C., more preferably 40° C., until the emulsion is formed (10-60 min). Usually, the amounts of the lipid extract and the ethanol are equal (by weight).

In step (ii), an aqueous solution containing up to 20% ascorbic acid (w/v) is added to the emulsion of (i), under mixing, and the mixture is transferred to a separatory device, for example, a separatory funnel. After about 30 min, distinct layers are formed, wherein the upper non-viscous liquid layers comprise the solvents (ethanol, water) and the neutral lipids (triglycerides) derived from the starting material, and the bottom distinct viscous liquid layer contains the desired phospholipids in enriched form.

In step (iii), the bottom viscous liquid layer is collected, water is added thereto, preferably 10-80, more preferably, 40 parts of water to 100 parts of the viscous liquid (vol), thus obtaining the phospholipids-enriched paste composition containing ascorbic acid.

The bottom viscous layer of (ii) in general contains about 75%-80% phospholipids and, after addition of water, the paste composition contains about 25%-60%, preferably 25-40%, more preferably about 30% phospholipids.

In one preferred embodiment, the lipid extract is from egg yolk.

Phospholipids are indispensable for the living organism and play an essential role both for health and in nutrition due to a great variety of different functions. They also prove to be perfect base substances for cosmetic products

The ascorbic acid used in the present invention is L-ascorbic acid, also known as vitamin C. Due to its antioxidant properties, vitamin C is considered to confer both antioxidant and photoprotection to skin against free radical attack and UV ray damage and has been used in recent years as an active ingredient in cosmetic compositions such as moisturizing cream, anti-aging cream, anti-wrinkle cream, sunscreen cream, and for stimulating collagen production. The terms “ascorbic acid” and “vitamin C” are sometimes used interchangeably herein.

The combination of phospholipids in high content and ascorbic acid makes the paste composition of the present invention a most suitable base for incorporation in cosmetic and nutraceutical compositions.

Thus, in another aspect, the present invention relates to a cosmetic product comprising a paste composition of the invention and a cosmetically acceptable carrier.

In the cosmetic product of the invention, the phospholipids are present in the amount of about 2% to about 20% by weight, preferably 2% to 5%, more preferably 4%, and the ascorbic acid is present in the amount of about 0.1% to about 0.7% by weight, preferably 0.2% to 0.5%, more preferably 0.3%. The LEC 40 paste composition as defined hereinafter in the cosmetic product may consist of 1% by weight of the total weight of the product.

The cosmetic product of the invention may further comprise one or more skin care active ingredients that may be water-soluble or oil-soluble.

Examples of active ingredients that may be used include, without being limited to, vitamins and vitamin analogs, herbal extracts, plant oils, color agents, glutathione, lipoic acid, Coenzyme Q10, a coumarin, a chalcone or a flavonoid, or any other other active ingredients obtained from a natural or synthetic source suitable for cosmetic use, and mixtures thereof.

The vitamins or vitamin analogs, besides the vitamin C originating from the paste composition, are selected from the ground consisting of vitamins A, B, D, E, F, K, P, analogs thereof, and mixtures thereof. Examples include, without being is limited to, retinoids, such as retinal palmitate, and their derivatives, panthenol or provitamin B5 and its derivatives, tocopherols including vitamin E, vitamin F, and rutin (vitamin P1), and mixtures thereof.

The herbal extracts and plant oils that may be used include, without being limited to, Ribes nigrum (Black Currant) Seed Oil, Helianthus Annuus (sunflower) Seed Oil, Licorice root extract, Grape Seed extract, Basil Essential Oil, Borage Oil, camellia Sinensis Leaf extract, Eucalyptus Essential Oil, Evening Primrose Oil, Geranium Essential Oil, Grapefruit Essential Oil, Hippophae Oil, Iris Florentina Root extract, Lemon Essential Oil, Morus Nigra Root extract, Peppermint Essential Oil, Saxifraga Sarmentosa extract, Scutellaria Baicalensis Root extract, Silybum Marianum Fruit extract, Tea Tree Oil, Vitis Vinifera (grape) fruit extract, and mixtures thereof.

The active ingredients may also be a coumarin, a chalcone or, a flavonoid selected from the group consisting of flavans, flavanols, flavonols, flavones, flavanones, isoflavones, anthocyanidins, proanthocyanidins, and mixtures thereof.

The coloring agent may be a pigment or a colorant, including, but not limited to, Carmine, iron oxides, titanium dioxide, mica, chrome oxide/hydroxide, D&C Red 21 Aluminum Lake, D&C Red 7 Calcium Lake, D&C Green 6 Liposoluble, Aluminium Blue #1, and mixtures thereof.

The cosmetic product of the invention may further comprise one or more additives chosen from anionic, cationic, nonionic and amphoteric surfactants, such as C12-20 acid PEG-8 ester, PEG-40 stearate, potassium cetyl phosphate or sorbitan tristearate; antioxidants besides the ascorbic acid of the LEC 40, such as ascorbyl palmitate, BHT, tocopherol, sodium metabisulfite or sodium sulfite; skin-conditioning agents, such as glycerin, dimethicone, butylene glycol, biosaccharide gum-1, butyrospermum park II (shea butter), carrageenan, caprylyl glycol, coco-caprylate-caprate, cyclopentasiloxane, cyclotetrasiloxane, black currant seed oil, tocopheryl acetate, retinyl palmitateor extracts of roots, leaves and fruits of different plants; humectants, such as PEG-8; film formers, such as sodium polyacrylate or C₃₀₋₄₅ alkyl cetearyl dimethicone crosspolymer; emulsion stabilizers, such as Carbomer, cetyl alcohol or glyceryl acrylate/acrylic acid copolymer; emulsifying agents, such as glyceryl stearate; chelating agents, such as disodium EDTA or citric acid; pH adjusters, such as sodium hydroxide; opacifying agents, such as titanium dioxide; preserving agents, such as phenoxyethanol or parabens (e.g. methyl-, ethyl-, propyl-, butyl- and isobutyl-paraben); antifoaming agents, such as ethanol; emollients, lubricants, fragrances, perfumes, colorants, and any other additives conventionally used in cosmetic compositions, and mixtures thereof.

The cosmetic product of the invention may contain one or more of the active ingredients in microencapsulated form. This is particularly important for unstable active ingredients used in cosmetics.

The cosmetic product of the invention may be in any suitable form, including, but not limited to, in the form of a cream, an emulsion, a mousse, a gel, a lotion, or a powder.

Thus, the paste composition of the invention can be incorporated into cosmetic products including, but not limited to, nutrient creams, eye creams, skin softeners, nutrient emulsions, massage creams, cellulite creams, cellulite lotions, anti-cellulite creams, body lotions, body creams, body milks, moisturizing creams, moisturizing lotions, dry skin lotions, dry skin creams, suntan compositions, and make-up cosmetics, such as foundations, blushers, eye shadows, lipsticks, etc., that are used to give colour to skin and lips. In particular, the cosmetic products are suitable as anti-aging agents.

Due to the high nutritive value of the phospholipids and of vitamin C, the paste composition of the invention is suitable as a dietary or food supplement for addition to food as such or as a component of nutraceutical products.

Thus, the present invention also provides a nutraceutical product or a dietary supplement comprising the paste composition of the invention, optionally together with other nutrients, such as vitamins, minerals, fatty acids, particularly of the n-3 and n-6 series, amino acids, sugars, and mixtures thereof. These products may also contain at least one member of the group consisting of alimentary auxiliary substances, sweetening agents, binding agents, taste agents, and flavoring agents, and are preferably solid, more preferably for oral ingestion, for example in the form of tablets, soft gelatine capsules, capsules, powders, and emulsions.

The invention will now be illustrated by the following non-limiting examples.

### EXAMPLES

Materials. Ovothin 120 (Cargill Inc) comprising 22-25% egg phospholipids (of which 60% phosphatidylcholine, 25% phosphatidylethanolamine, 15% others), 60-70% neutral lipids (of which 85% triglycerides, 5% mono- and diglycerides, 6-8% cholesterol, 2-4% others).

### Example 1

### Preparation of Egg Yolk Phospholipids-Enriched Paste Composition Containing Ascorbic Acid

1 kg Ovothin 120 was mixed with 1 kg 95% ethanol at 40° C. To the formed emulsion, 100-500 ml, preferably 350 ml of water containing 17.5 g ascorbic acid (Sigma, Israel) were added with mixing and then transferred to a reparatory funnel. After 30 minutes, three distinct layers were formed: the upper layer contained ethanol-water (1200 ml), the middle layer contained predominantly neutral lipids (triglycerides) (700 ml), and the bottom layer (500 ml) predominantly phospholipids.

The viscous bottom layer was 55% solid (of which 50-70% are phospholipids) and 45% liquid. The bottom layer was separated and water was added thereto (about 40 ml water for about 100 ml viscous liquid), thus resulting in a uniform paste containing 25-40%, preferably 30%, phospholipids and 0.5-1.5% ascorbic acid, having the consistency of mayonnaise, a dark yellow color, and the characteristic smell of lecithin. This paste, herein designated “LEC 40”, is highly enriched in phospholipids (contains more than 50% posphatidylcholine) and can be used as a component of cosmetic and nutraceutical compositions and dietary supplements.

### Example 2

### Eye Cream Comprising LEC-40

An anti-aging eye cream comprising 1% w/w of LEC-40 as emulsifying agent was prepared containing the following main ingredients:

| Phase | % w/w | Ingredient |
|---|---|---|
| A | 12.8 | Butylene glycol |
| | 3 | Glycerin |
| | 0.6 | PEG-8 |
| B | 0.4 | Caprylyl glycol |
| C | 6,5 | Coco-caprylate/caprate |
| | 3.2 | Glyceryl stearate |
| | 1.3 | Cetyl alcohol |
| | 1.1 | C₁₂₋₂₀ acid PEG-8 ester |
| | 1.0 | Lecithin LEC-40 |
| | 0.6 | PEG-40 stearate |
| D | 2.4 | Cyclopentasiloxane |
| E | 4.4 | Fragrances, Antioxidants, Preservatives |

A dispersion of the components of Phase A in water was heated to 70-80° C. and homogenized for 45-50 minutes. After addition of the component of Phase B, the dispersion was homogenized for additional 10 minutes. The ingredients of phase C were dissolved at 70-80° C. and added to the dispersion of combined Phases A and B. The resulting dispersion was homogenized under partial vacuum for 10 minutes, followed by addition of the Phase D component at 40° C. and the phase E component at 37° C. Vacuum was reinforced at 28° C. The cream obtained was packaged in suitable containers.

### Example 3

### Anti-Age Cream Comprising LEC-40

An anti-aging cream comprising 1% w/w of LEC-40 as emulsifying agent was prepared containing the following main ingredients:

| Phase | % w/w | Ingredient |
|---|---|---|
| A | 10 | Glycerin |
| | 6.3 | Butylene glycol |
| B | 0.1 | Caprylyl glycol |
| C | 11.6 | Caprylic capric triglyceride |
| | 3.5 | Coco-caprylate/caprate |
| | 3 | Dimethicone |
| | 2.2 | Glyceryl stearate |
| | 2.2 | Cetyl alcohol |
| | 2.0 | PEG-40 stearate |
| | 1.7 | Butyrospermum (Shea Parkii Butter) |
| | 1.4 | Potassium Cetyl Phosphate |
| | 1.1 | Mica |
| | 1.0 | Lecithin LEC-40 |
| D | 5.9 | Cyclopentasiloxane |
| E | 4.4 | Fragrances, Antioxidants, and Preservatives |

The components of phase A were dispersed in water, followed by heating to 70-80° C., and homogenized for 45 minutes. The phase B ingredient was added and the dispersion homogenized for additional 10 minutes. The ingredients of phase C were dissolved at 80° C. and added to combined phases A and B. The resulting dispersion was homogenized under partial vacuum for 10 minutes. Phase D was added at 40° C., followed by phase E at 37° C. Vacuum was reinforced at 28° C. The resulting anti-aging cream was packaged in suitable containers.

Other cosmetic products according to the invention can be prepared in a similar way.

1. A paste composition comprising at least 25% phospholipids by weight in combination with at least 0.5% ascorbic acid by weight.

2. The paste composition according to claim 1, comprising about 25% to about 60% phospholipids by weight and about 0.5% to about 1.5% ascorbic acid by weight, based on the total weight of said composition.

3. The paste composition according to claim 2, comprising about 30% to about 40% by weight phospholipids and about 1% by weight ascorbic acid.

4. The paste composition according to claim 1, wherein the phospholipids are obtained from an egg yolk lipid extract.

5. The paste composition according to claim 1, wherein at least 50% of said phospholipids is phosphatidylcholine.

6. A method for the preparation of a paste composition according to claim 1, comprising the steps:
(i) fanning an emulsion of a lipid extract in ethanol;
(ii) adding water containing ascorbic acid to said emulsion of (i), under mixing, and transferring to a separatory device, whereby three different layers are formed, the viscous bottom layer consisting mainly of phospholipids; and
(iii) collecting said bottom viscous liquid layer of (ii) and adding water thereto, thereby obtaining the desired paste composition comprising at least about 25% phospholipids and at least about 0.5% ascorbic acid.

7. The method according to claim 6, wherein said lipid extract is obtained from egg yolk.

8. A cosmetic product comprising a paste composition according to claim 1 and a cosmetically acceptable carrier.

9. The cosmetic product according to claim 8, further comprising one or more water-soluble or oil-soluble skin care active ingredients.

10. The cosmetic product according to claim 9, wherein said skin care active ingredients are selected from the group consisting of vitamins and vitamin analogs, herbal extracts, plant oils, coloring agents, glutathione, lipoic acid, Coenzyme Q10, a coumarin, a chalcone or a flavonoid, or any other active ingredients obtained from a natural or synthetic source suitable for cosmetic use, and mixtures thereof.

11. The cosmetic product according to claim 10, wherein said vitamins or vitamin analogs are selected from the group consisting of vitamins A, B, D, E, F, K, P, analogs thereof, and mixtures thereof.

12. The cosmetic product according to claim 11, wherein said vitamins and vitamin analogs include retinoids, such as retinol palmitate, and their derivatives, panthenol or provitamin B5 and its derivatives, tocopherols including vitamin E, vitamin F, and rutin (vitamin P1), and mixtures thereof.

13. The cosmetic product according to claim 8, further comprising one or more additives chosen from anionic, cationic, nonionic and amphoteric surfactants, antioxidants, skin conditioning agents, humectants, film formers, emollients, lubricants, fragrances, perfumes, preserving agents, emulsion stabilizers, emulsing agents, pH adjusters, opacifying agents, audioactive agents, colorants.

14. The cosmetic product according to claim 9, wherein one or more of the active ingredients are in microencapsulated form.

15. The cosmetic product according to claim 8, wherein the product is in the form of a cream, an emulsion, a mousse, a gel, a lotion, or a powder.

16. A nutraceutical product comprising the paste composition according to claim 1.

17. A dietary supplement comprising a paste composition according to claim 1.

18. The nutraceutical product or dietary supplement according to claim 16, further comprising other nutrients, such as vitamins, minerals, fatty acids, amino acids, sugars, and mixtures thereof.
